(19) United States
(12) Patent Application Publication
(10) Pub. No.: US 2011/0104089 A1
(43) Pub. Date: May 5, 2011

(54) COMPOSITIONS COMPRISING FRAGRANCE SUBSTANCES AND NEOPENTYL GLYCOL DIISONONANOATE

(75) Inventors: Ingo Wöhrle, Bremen (DE); Oskar Koch, Gottingen (DE); Manfred Meier, Furstenberg (DE); Gerhard Schmaus, Hoxter-Bosseborn (DE)

(73) Assignee: SYMRISE AG, Holzminden (DE)

(21) Appl. No.: 12/938,137

(22) Filed: Nov. 2, 2010

(30) Foreign Application Priority Data
Nov. 2, 2009 (EP) 09 174 780.8

(51) Int. Cl. A61K 8/97; A61Q 17/04; A61Q 15/00; A61Q 19/00; A61Q 5/00; A61K 8/36; A61K 8/37; A61Q 13/00

(52) U.S. Cl. 424/60; 424/725; 424/65; 512/26; 512/11

(57) ABSTRACT

The invention relates to the use of neopentyl glycol diisononanoate (CAS No. 27841-07-2) as a fixative for perfume oils or as a fixative for (top note) fragrance substances. The invention also relates to certain compositions containing neopentyl glycol diisononanoate and one or more top note fragrance substances. Furthermore, the invention relates to a method for producing such compositions and (cosmetic) products containing a composition according to the invention. Additionally the invention relates to a method for imparting, intensifying or modifying an odor on the (human) skin. A particular aspect of the present invention relates to the improved adherence of volatile fragrance substances to (human) skin and/or (human) hair. Apart from this the invention relates to a combination (kit) comprising neopentyl glycol diisononanoate and one or more top note fragrance substances.

The invention relates to the use of neopentyl glycol diisononanoate (CAS No. 27841-07-2) as a fixative for perfume oils or as a fixative for (top note) fragrance substances. The invention also relates to certain compositions containing neopentyl glycol diisononanoate and one or more top note fragrance substances. Furthermore, the invention relates to a method for producing such compositions and (cosmetic) products containing a composition according to the invention. Additionally the invention relates to a method for imparting, intensifying or modifying an odor on the (human) skin. A particular aspect of the present invention relates to the improved adherence of volatile fragrance substances to (human) skin and/or (human) hair. Apart from this the invention relates to a combination (kit) comprising neopentyl glycol diisononanoate and one or more top note fragrance substances.

In the area of fragrance substances and perfumery it is generally known that when fragrance substance compositions are used on the skin, as a result of the evaporation of the diluents or solvents there is a decrease in fragrance substances, in particular of the volatile top note, while the less volatile notes, in particular the so-called bottom notes, have a exceptional adherence to the skin and are given off from the skin over a long period and as a result their smell is perceptible for longer. The result of the more rapid evaporation of the top note is that over time a marked change results in the olfactory profile of fragrance substance compositions applied to the skin. A similar effect occurs when fragrance substance compositions are used on (human) hair. Here it is necessary to take into account that the adverse effect described is in particular perceived as problematic if a fragrance substance composition (fragrance substance mixture) is used as a leave-on-product, that is to say that it is to remain on the skin and/or the hair, in order to be given off from there over a longer period.

Adhesion and adherence within the context of the present text mean the adhesion of a fragrance substance to skin and hair, in particular the adhesion to human skin (except for the (oral) mucosa). So although the present invention with regard to the application of fragrance substance compositions also relates to the (oral) mucosa, it must nevertheless be stated that the significance of the present invention concerning the skin aspect (excluding the oral mucosa) and/or hair (in particular human hair) is of much greater relevance, for when a fragrance substance composition is applied to the (oral) mucosa the negative evaporation effect is normally superimposed and dominated by washing away effects (in the area of the oral mucosa, for example supported by the effect of saliva).

In perfumery practice many trials have been performed in the past to try and extend the adherence or the perceptibility, in particular of the top note, of fragrance substance compositions, in particular on human skin and thus to achieve a certain odor “profile stability”.

A fixative increases the adherence of fragrance substances, e.g. by lowering their vapor pressure. Here fixatives mean substances which allow a delayed release over time of the perfume oil components, for example on skin and/or hair, thus ensuring a longer-lasting impression of the fragrance.

Particularly suitable here are fixatives that are odorless or have a low inherent odor, so that they do not alter the odor impression of fragrance substances, fragrance substance mixtures and perfume oils.

The use of fixatives has been described many times, e.g. the use of individual substances or combinations of substances (fragrance substances), which are added to highly volatile fragrance substances or fragrance substance mixtures, in order to reduce their speed of evaporation (see U.S. Pat. No. 6,737,396).

In IP.COM #000033581D (published in January 2005) the use of hydroxyalkyl urea derivatives, in particular hydroxyethyl urea, is described, by means of which the adhesion of perfumes in cosmetic applications, in particular aqueous-ethanolic formulations, on skin and hair is prolonged.

In U.S. Pat. No. 3,939,099 the use of film-formers is described, which dissolve in a water/ethanol mixture and are miscible with fragrance substance (mixtures). Quoted examples are ionic and non-ionic derivatives of water-soluble polymers such as for example polyvinylpyrrolidone derivatives, quaternary polyvinylpyrrolidones with molecular weights in the range of 50,000-1,000,000, cationic cellulose derivatives and similar. During the evaporation of ethanol a film forms on the skin in which the fragrance substances are stored.

U.S. Pat. No. 6,210,688 describes the formation and use of an odorless polymer film on the skin (based on vinyl ether copolymers, polyacrylates, methacrylates, polyesters, polyfluorohydrocarbons, polysaccharides), to which a perfume is then applied. In this way “reactions” with the skin are prevented.

FR 2 747 306 describes the use of polymer hydrocarbons (polyethylenes with molecular weights of 3,000-30,000). Since these polymers are insoluble in ethanol/water, a corresponding product such as for example eau de cologne, eau de toilette or eau de partum will be turbid.

WO 2004/098556 describes a novel sprayable and clear perfume formulation, which is characterized by an increased surface tension or reduced contact area following application, which is achieved through the use of an effective quantity of a polymer. By means of the smaller contact area the perfume oil remaining after evaporation of the ethanol is concentrated in a smaller area, from which the fragrance substances evaporate more slowly.

Film-formers and/or polymers have the disadvantage that they reduce the speed of evaporation of not only the top note, but also all other less volatile fragrance substances, so that the overall intensity is markedly reduced. Furthermore, film-formers can cause an unpleasant tacky or tightening feeling on the skin.

EP 0 181 401 and EP 0 857 481 describe perfume preparations in gel form, in which the diffusion and thus also the evaporation of fragrance substances is reduced. However, the proposed gel formation is particularly unsuitable for some application areas, such as for example fine perfumery or products in lotions or spray form, since the preparations in gel form that they describe are too viscous.

EP 1 872 831 describes certain ethanolic fragrance substance compositions containing deliquescent substances for imparting, intensifying or modifying an odor, in particular here improving the adherence of ethanolic fragrance substance compositions to (human) skin and/or (human) hair.

The fixatives sought should meet the above requirements and be easily miscible with as many as possible of the current (perfumery) fragrance substances and perfume oils as well as being able to be incorporated in a number of cosmetic formulations.

It was therefore a primary object for the invention to indicate a(n alternative) fragrance substance composition, in which the fragrance substances used, preferably fragrance substances which form the top note of a fragrance, have a long (longer compared with conventional fragrance substance compositions) adhesion (adherence), in particular to (human) skin (here skin excludes the mucosa) and/or (human) hair. In doing so the disadvantages of previous fragrance substance compositions described above should be avoided as far as possible.

The primary object posed is achieved according to the invention by a composition comprising (or consisting of):

(A) neopentyl glycol diisononanoate,

and

(B) one or more fragrance substances, in particular a fragrance substance mixture comprising 2, 3, 4, 5, 6, 7, 8, 9, 10 or more fragrance substances,

wherein component (B)

(B) (i) contains one, 2, 3, 4, 5 or more fragrance substances with a molecular weight in the range of 100 g/mol-175 g/mol (top note), preferably with a molecular weight in the range of 110 g/mol-165 g/mol, preferably in the range of 115 g/mol-160 g/mol, particularly preferably in the range of 120 g/mol-155 g/mol,

and

wherein component (A) is preferably contained in a quantity that fixes the, a plurality of or all fragrance substances of component (B), preferably in a quantity that fixes the, a plurality of or all fragrance substances of component (B) (i).

A fixing quantity of component (A) means a quantity of component (A) which is sufficient to bring about a fixing of one, a plurality of or all fragrance substances of component (B).

Preferably, the mass ratio of component (A) to the total mass of component (B) is in the range of 1:20-200:1, preferably in the range of 1:12-100:1, more preferably in the range of 1:6-50:1, particularly preferably in the range of 1:5-33:1, and quite particularly preferably in the range of 1:2-25:1.

When the mass ratios of components (A) and (B) specified (as preferred) are employed in a composition according to the invention or in a cosmetic (topical) product according to the invention, the fixing effect of component (A) is brought about on component (B).

Surprisingly, the compositions according to the invention demonstrate a fixing of the, a plurality of or all fragrance substance(s) of component (B) through component (A), in particular when the mass ratios specified as preferred are used. Through an effective fixing quantity of component (A), which acts as fixative for one, a plurality of or all fragrance substances of component (B), an improved and extended adhesion (adherence) of component (B) is achieved, in particular to (human) skin and/or (human) hair. This also applies to cosmetic (topical) products according to the invention, which preferably contain a sensorially (olfactorily) effective quantity of a composition according to the invention.

EP 2 014 749 relates to base oils as a component of coolants for carbon dioxide operated refrigerators. The base oils used there relate to primary esters of polyhydric alcohols and C14-C22 branched-chain carboxylic acids in mixture with other esters; inter alia two special base oils with low proportions of neopentyl glycol diisononanoate are described (base oil 4 and base oil 26). These base oils contain no fragrance substances.

In preferred embodiments a composition according to the invention or a cosmetic (topical) product according to the invention is free from mixtures of neopentylglycol-di-n-dodecanate and neopentyl glycol diisononanoate in the weight ratio 1.4:15 and/or from neopentyl glycol-di-n-tetradecanate and neopentyl glycol-diisononanoate in the weight ratio 2:15 and/or from neopentyl glycol-di-n-pentadecanate and neopentyl glycol diisononanoate in the weight ratio 0.7:15. In further preferred embodiments a composition according to the invention or a cosmetic (topical) product according to the invention is free from neopentyl glycol-di-n-dodecanate and/or neopentyl glycol-di-n-tetradecanate and/or neopentyl glycol-di-n-pentadecanate. In further preferred embodiments a composition according to the invention or a cosmetic (topical) product according to the invention is not a base oil or coolant oil as described in EP 2 014 749. In a further preferred embodiment a composition according to the invention or a cosmetic (topical) product according to the invention is free from C1-C3 fluorohydrocarbons and/or esters of polyhydric alcohols and C14-C22 branched-chain carboxylic acids.

WO 94/13764 discloses mixtures of neopentyl glycol diisononanoate and 1,1,1-trifluorethane as coolants. In preferred embodiments a composition according to the invention or a cosmetic (topical) product according to the invention is free from 1,1,1-trifluorethane.

U.S. Pat. No. 5,202,044 and U.S. Pat. No. 5,395,444 describe coolants, which have a difluoromethane base. The coolants described there contain certain esters, wherein neopentyl glycol diisononanoate is also mentioned. The coolants described there can also contain 0.001-2 wt % of metal deactivators, with reference to the quantity of esters used. The list of metal deactivators includes substances that have an odor such as ethylene diamine and diethyl malonate, in particular also acetyl acetone and acetoacetic esters (ethyl-3-oxobutanoate). U.S. Pat. No. 5,202,044 and U.S. Pat. No. 5,395,444 do not expressly disclose a mixture of neopentyl glycol diisononanoate and odorous metal deactivators. In preferred embodiments a composition according to the invention or a cosmetic (topical) product according to the invention is free from difluoromethane. In further preferred embodiments a composition according to the invention or a cosmetic (topical) product according to the invention is either free from ethylenediamine, diethyl malonate, acetyl acetone (which are not perfumery fragrance substances according to the definition below) and acetoacetic ester (which is a perfumery fragrance substance according to the definition below) or the composition according to the invention or the cosmetic according to the invention contains one (further), preferably two, three, four, five or more (further) perfumery fragrance substances (as defined below).

In a further preferred embodiment a composition according to the invention or a cosmetic product according to the invention contains no fluorochlorohydrocarbons, in particular no C1-C2 fluorochlorohydrocarbons, and/or no C1-C3 fluorohydrocarbons, in particular not those (expressly) described in the above documents. Such fluorochlorohydrocarbons and fluorohydrocarbons are odorless and as such are not considered to be fragrance substances, in particular not perfumery fragrance substances according to the definition below.

In Kongop Hwahak 2001, 12, 334-339 the hydrolysis speeds of various esters, including neopentyl glycol diisononanoate, were investigated. The hydrolysis catalyst used there was p-toluol sulfonic acid.

DE 1912486 describes the esterification of neopentyl glycol with a mixture of n-nonanic acid (pelargonic acid) and isononanic acid (3,5,5-trimethylhexanoic acid) to 2,2-dimethylpropan-1,3-diol-3,5,5-hexanate-pelargonate in the presence of concentrated sulfuric acid in cumol as the water entrainer agent. It is not known here if in the reaction mixture neopentyl glycol diisononanoate was also formed. DE 1912486 states concerning neopentyl glycol diisononanoate merely that it has poorer lubricant properties than 2,2-mimethylpropan-1,3-diol-3,5,5-hexanate-pelargonate. In preferred embodiments a composition according to the invention or a cosmetic (topical) product according to the invention is not a (reaction) mixture as described in DE 1912486. As a precaution in further preferred embodiments a composition according to the invention or a cosmetic (topical) product according to the invention is free from 2,2-dimethylpropan-1,3-diol-3,5,5-hexanate-pelargonate, cumol and/or concentrated sulfuric acid.

JP 52131513 discloses the use of neopentyl glycol diisononanoate in cosmetics for skin and hair. These cosmetics can contain perfume, but no details of the quantities of perfume to be used are provided. Neither are any individual fragrance substances and no olfactory effects described there. JP 52131513 also discloses the production of neopentyl glycol diisononanoate by means of azeotropic esterification of neopentylglycol and isononanic acid in xylol in the presence of n p-toluol sulfonic acid.

JP 2008-162965 describes cleansing cosmetic crèmes, which generally contain 50-95 wt % of neopentyl glycol diisononanoate. The above document also states that the cleansing cosmetic crèmes described there may contain normal additives, with perfume being stated as one additive amongst others. Nothing is said there about the perfume, and in particular no individual fragrance substances are specified. Furthermore, no olfactory effects are described there and no details of quantities are provided for the optional perfume additive. JP 2008-162965 also describes the production of neopentyl glycol diisononanoate by means of azeotropic esterification of neopentyl glycol and isononanic acid in toluol in the presence of p-toluol sulfonic acid.

The methods described in the above documents for the esterification of neopentyl glycol and isononanic acid for the production of neopentyl glycol diisononanoate use aromatic solvents in the azeotropic esterification as the water entrainer.

Toluol, xylols and cumol are not perfumery fragrance substances according to the definition below. In particular, the reactions mixtures described in each of the above documents are not the subject matter of the present invention; all the more so as these solvents are extremely hazardous to health and thus are not suitable for use in the area of the present invention, in particular not for use in cosmetic (topical) products. In preferred embodiments therefore compositions and cosmetic (topical) products according to the invention are free from toluol, xylolene and cumol, preferably free from aromatic hydrocarbons with a molar mass of less than 125 g/mol.

Alternatively and preferably in addition, compositions and cosmetic (topical) products according to the invention are free from p-toluol sulfonic acid and free from (concentrated) sulfuric acid (H₂SO₄).

Alternatively and preferably in addition, compositions and cosmetic (topical) products according to the invention are free from isononanic acid and/or free from pelargonic acid, which are not perfumery fragrance substances according to the definition below.

Component (A)

Neopentyl glycol diisononanoate [CAS No. 27841-07-2; 2,2-dimethyl-1,3-propandiol diisononanoate, diesters of 2,2-dimethyl-1,3-propandiol and of 3,5,5-trimethylhexanoic acid (isononanic acid); 2,2-dimethyl-1,3-propandiol-di-3,5,5-trimethylhexanate] has the following structural formula (I):

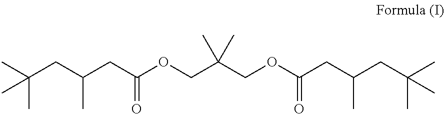

Neopentyl glycol diisononanoate can by way of example be prepared using normal chemical or biotechnological esterification methods known to a person skilled in the art by conversion of neopentyl glycol and 3,3,5-trimethyl hexanoic acid. The compound of formula (I) is also commercially available.

Component (A) of a composition according to the invention is preeminently suitable for the object posed, since neopentyl glycol diisononanoate has no, or in any case only a very slight, inherent odor. Component (A) is obviously not part of component (B).

Neopentyl glycol diisononanoate also imparts a pleasant feeling to the skin, such that compositions according to the invention are preeminently suitable for application to the (human) skin and for incorporation in and as part of cosmetic (topical) products.

Furthermore, because of its good spreading ability on the (human) skin, neopentyl glycol diisononanoate ensures that the fragrance substance(s) of component (B) of a compositions according to the invention or a cosmetic (topical) product according to the invention are distributed evenly over the (human) skin. Similarly any (preferably lipophilic) (active) substances present in such compositions or products according to the invention are distributed evenly over the skin.

Component (B)

Examples of fragrance substances of component (B), and similarly of the (preferred) components (B*), (B) (i) and (B) (ii), will be known to a person skilled in the art and can be found for example in S. Arctander, Perfume and Flavor Materials, Vol. I and II, Montclair, N.J., 1969, author's edition or H. Surburg, J. Panten, “Common Fragrance and Flavor Materials”, 5^{th}. Ed., Wiley-VCH, Weinheim 2006.

Component (B) is preferably a fragrance substance mixture, preferably a fragrance substance mixture comprising 3, 4, 5, 6, 7, 8, 9, 10 or more fragrance substances, more preferably perfumery fragrance substances, in particular a perfume oil.

Preferred fragrance substances of component (B) are perfumery fragrance substances, which in turn are preferably selected from group (B*) consisting of the group of individual compounds disclosed as fragrance substances according to
S. Arctander, Perfume and Flavor Materials, Vol. I and II, Montclair, N.J., 1969, author's edition (“Arctander”);
H. Surburg, J. Panten, “Common Fragrance and Flavor Materials”, 5^{th}. Ed., Wiley-VCH, Weinheim 2006 (“Surburg”),

and
the individual compounds designated as fragrance substances within the context of the present text.

If in individual cases there should be a lack of correspondence or a contradiction between these three sources, the present text shall take precedence over “Arctander” and “Surburg”. If in individual cases there should be a lack of correspondence or a contradiction between “Surburg” and “Arctander”, “Surburg” shall take precedence over “Arctander”.

In a preferred embodiment the present invention relates to a composition comprising (or consisting of):

(A) neopentyl glycol diisononanoate,

and

(B) one or more fragrance substances from group (B*) as defined above, preferably a fragrance substance mixture comprising 2, 3, 4, 5, 6, 7, 8, 9, 10 or more fragrance substances from group (B*), wherein (B*)

(B) (i) contains one, 2, 3, 4, 5 or more fragrance substances with a molecular weight in the range of 100 g/mol-175 g/mol (top note), preferably with a molecular weight in the range of 110 g/mol-165 g/mol, preferably in the range of 115 g/mol-160 g/mol, particularly preferably in the range of 120 g/mol-155 g/mol,

and

wherein component (A) is preferably contained in a quantity that fixes the, a plurality of or all fragrance substances of component (B*), preferably in a quantity that fixes the, a plurality of or all fragrance substances of component (B) (i).

The mass ratio of component (A) to the total mass of component (B*) is preferably in the range of 1:20-200:1, preferably in the range of 1:12-100:1, more preferably in the range of 1:6-50:1, particularly preferably in the range of 1:5-33:1, and quite particularly preferably in the range of 1:2-25:1.

Other preferred compositions according to the invention are those in which component (B), preferably component (B*),

(B) (i) contains one or more fragrance substances with a molecular weight in the range of 100 g/mol-175 g/mol (top note), preferably with a molecular weight in the range of 110 g/mol-165 g/mol, preferably in the range of 115 g/mol-160 g/mol, particularly preferably in the range of 120 g/mol-155 g/mol,

and

(B) (ii) contains one, 2, 3, 4, 5 or more fragrance substances with a molecular weight of greater than or equal to 190 g/mol (bottom note), preferably with a molecular weight in the range of 190 g/mol-300 g/mol, preferably with a molecular weight in the range of 195 g/mol-290 g/mol, and particularly preferably in the range of 200 g/mol-275 g/mol.

In a preferred embodiment a composition according to the invention contains at least 2, 3, 4, 5 or more fragrance substances of component (B) (i), and/or at least 2, 3, 4, 5 or more fragrance substances of component (B) (ii).

Compositions according to the invention, containing one or more fragrance substances of component (B) (i) (referred to as preferred), in particular in the proportions indicated in the following, and one or more fragrance substances of component (B) (ii), also demonstrate an olfactory harmonization and rounding off, resulting in a more elegant and thus olfactorily more valuable olfactory impression. This applies in particular to musk fragrance substances and other bottom notes, referred to in the following as preferred, of component (B) (ii), in particular in the proportions indicated in the following.

Here, the (preferred) fragrance substances of components (B), (B*), (B) (i) and (B) (ii), where applicable, can be present in the form of their respective diastereomers, enantiomers and/or double bond isomers. Thus these can be present in the form of (E)/(Z)-isomers, any mixture of the enantiomers, in particular racemate, or also any mixture of the corresponding diastereomers.

Component (B) (i)

The fragrance substances of component (B) (i) are to be regarded as top notes of a composition according to the invention or of a cosmetic product according to the invention. The top note determines the initial odor (first phase) of a fragrance substance mixture or a perfume oil.

The molecular weight of the fragrance substances of component (B) (i) is in the range of 100 g/mol-175 g/mol (top note), preferably in the range of 110 g/mol-165 g/mol, preferably in the range of 115 g/mol-160 g/mol, and particularly preferably in the range of 120 g/mol-155 g/mol.

Our own investigations have also shown that such fragrance substances of component (B) (i) are fixed particularly well by component (A), where selected from the group consisting of:
the group of (preferably non-aromatic) alcohols, here in turn preference is for terpene alcohols and aliphatic alcohols,
the group of (preferably non-aromatic) ketones,
the group of (preferably non-aromatic) aldehydes,
the group of (preferably non-aromatic) nitriles, and
the group of (preferably non-aromatic) esters.

The fragrance substances referred to in the following as “non-aromatic” in connection with the present invention are those which in the structural sense contain no aromatic ring, i.e. no phenyl group.

Component (B) (i) preferably comprises a composition according to the invention (in particular in one of the embodiments referred to as preferred) one or more fragrance substances from the group consisting of:

(B) (i) n-Heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol (2,6-dimethyl-7-octen-2-ol), menthone, isomenthone, 2,6-dimethyl-5-hepten-1-al (Melonal), 3-hexenyl methyl carbonate, benzaldehyde, linalool, tetrahydrolinalool, tetrahydrogeraniol, citral, neral, geranial, benzyl alcohol, benzyl acetate, methyl cinnamate, p-anisaldehyde, menthol, isoamyl acetate, isoamyl butyrate, cis-3-hexenyl acetate, trans-3-hexenyl acetate, hexyl acetate, butyl butyrate, citronellol, nerol, geraniol, 2-phenylethyl alcohol, methyl benzoate, methyl anthranilate, styrallyl acetate, 2,4-dimethyl-3-cyclohexene carboxaldehyde (vertocitral), acetophenone, p-methyl acetophenone, cinammic alcohol, cinammic aldehyde, 3-phenyl propanol (dihydrocinammic alcohol), 2-methyl-1-phenyl-2-propanol (alpha,alpha-dimethyl phenethyl carbinol), anethole, anisalcohol, heliotropin, acetanisole (p-methoxy acetophenone) and agrunitrile (3,7-dimethyl-6-octen-1-nitrile).

Preference is for the following fragrance substances of component (B) (i), which are “non-aromatic” in the structural sense, since with these a higher retention has been achieved: n-heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol (2,6-dimethyl-7-octen-2-ol), menthone, isomenthone, 2,6-dimethyl-5-hepten-1-al (Melonal), 3-hexenyl methyl carbonate, linalool, tetrahydrolinalool, tetrahydrogeraniol, citral, neral, geranial, menthol, isoamyl acetate, isoamyl butyrate, cis-3-hexenyl acetate, trans-3-hexenyl acetate, hexyl acetate, butyl butyrate, citronellol, nerol, geraniol, 2,4-dimethyl-3-cyclohexene carboxaldehyde (vertocitral) and agrunitrile (3,7-dimethyl-6-octen-1-nitrile).

Particular preference is for the following fragrance substances of component (B) (i), since with these an even higher retention has been achieved: n-heptanol, camphor, alpha-pinene, beta-pinene, alpha-terpineol, limonene, 6-methyl-5-hepten-2-one, n-octanal, 3-hexenol, dihydromyrcenol (2,6-dimethyl-7-octen-2-ol), menthone, isomenthone, 2,6-dimethyl-5-hepten-1-al (Melonal), 3-hexenyl methyl carbonate, linalool, tetrahydrolinalool, tetrahydrogeraniol, citral, neral, geranial, menthol, isoamyl acetate, isoamyl butyrate, cis-3-hexenyl acetate, hexyl acetate, citronellol, nerol, geraniol, 2,4-dimethyl-3-cyclohexene carboxaldehyde (vertocitral) and agrunitrile (3,7-dimethyl-6-octen-1-nitrile).

Greatest preference is for the following fragrance substances of component (B) (i), since with these the highest retention has been achieved:

n-heptanol, camphor, limonene, dihydromyrcenol (2,6-dimethyl-7-octen-2-ol), linalool, tetrahydrolinalool, citronellol, geraniol and agrunitrile (3,7-dimethyl-6-octen-1-nitrile).

When the fragrance substances from group (B) (i) referred to as (particularly) preferred are used the stated effects are particularly pronounced.

The total quantity of the fragrance substance(s) (referred to as preferred) of component (B) (i) (top note) of a composition preferred according to the invention or a cosmetic product preferred according to the invention is 5-80 wt %, particularly preferably 10-70 wt %, in each case in relation to the total weight of component (B), preferably in each case in relation to the total weight of component (B*).

Component (B) (ii)

The fragrance substances of the optional component (B) (ii) are to be regarded as bottom notes of a composition according to the invention or a cosmetic product according to the invention. The bottom note determines the after odor of a fragrance substance mixture or a perfume oil.

The molecular weight of the fragrance substances of component (B) (ii) is greater than or equal to 190 g/mol and is preferably in the range of 190 g/mol-300 g/mol, more preferably in the range of 195 g/mol-290 g/mol, and most preferably in the range of 200-275 g/mol.

With such fragrance substances of component (B) (ii) the described effects of the present invention can be achieved at their most pronounced and clearest.

Preferred compositions according to the invention are those in which component (B) (ii) contains one, two, three or more fragrance substances, selected from the group consisting of:
musk fragrance substances,

alpha-n-amylcinnamaldeyde (MW=202.30), alpha-iso-amylcinnamaldehyde (MW=202.30), alpha-n-hexylcinnamaldehyde (MW=216.32), alpha-iso-hexylcinnamaldehyde (MW=216.32), benzyl salicylate (MW=228.25), cis-3-hexenyl salicylate (MW=220.27), isoamyl salicylate (MW=208.26), hexyl salicylate (MW=222.28), 2-methyl-3-(4-tert-butylphenyl)propanal (MW=204.31; Lilial®), 2-methyl-3-(4-isopropylphenyl)propanal (MW=190.28, cyclamen aldehyde), 2,3,8,8-tetramethyl-1,2,3,4,5,6,7,8-octahydro-2-naphthalenyl methyl ketone (MW=234.38, Iso E Super®), 1-[1,2,3,4,6,7,8,8a-octahydro-1,2,8,8-tetramethyl naphtalen-2-yl]ethanone (MW=234.38, Iso E Super®), methyl dihydrojasmonate (MW=226.32, Hedione®), linalyl acetate (MW=196.29), ethyllinalyl acetate (MW=210.31), nerolidol (MW=222.37), farnesol (MW=222.37), cedryl methyl ether (MW=236.40, Cedramber), cedryl methyl ketone (MW=246.39), cedryl acetate (MW=264.41), (4aR,5R,7aS,9R)-octahydro-2,2,5,8,8,9a-hexamethyl-4H-4-a,9-methanoazuleno(5,6-d)1,3-dioxol) (MW=278.44, Ambrocenide®), hexahydro-1′,1′,5′,5′-tetramethyl-spiro[1,3-dioxolan,2,8′(5′H)-[2H-2,4-a]-methanonaphthalene (MW=264.41 ethylenedioxy-3H-isolongifolane, Ysamber® K), 2-methyl-4-(2,2,3-trimethyl-3-cyclopenten-1-yl)butanol (MW=196.34, Brahmanol), 5-(2,2,3-trimethyl-3-cyclopenten-1-yl)-3-methylpentan-2-ol (MW=210.36, Sandalore®), 2-ethyl-4-(2,2,3-trimethyl-3-cyclopenten-1-yl)-2-buten-1-ol (MW=208.35, Sandranol®), 3-methyl-5-(2,2,3-trimethyl-3-cyclopenten-1-yl)-4-penten-2-ol (MW=208.35, Ebanol®), 3,3-dimethyl-5-(2,2,3-trimethyl-3-cyclopenten-1-yl)-4-penten-2-ol (MW=222.37, Polysantol®), 3-isocamphylcyclohexanol (MW=236.40, Sandel 80®), 1-(2,2,6-trimethylcyclohexyl)hexan-3-ol (MW=226.41, Timberol®), cyclododecyl methyl ether (MW=198.35, Palisandin), (ethoxymethoxy)cyclododecane (MW=242.41, Boisambrene Forte®), 1-methyl-4-(4-methyl-3-penten-1-yl)-3-cyclohexene carboxaldehyde (MW=206.33, Precyclemone B®), 4-(4-hydroxy-4-methylpentyl)-3-cyclohexene carboxaldehyde (MW=210.32, Lyral®), 2-methyl-4-(2,2,6-trimethyl-1-cyclohexen-1-yl)-2-butenal (MW=206.33, Boronal), decahydro-beta-naphthyl acetate (MW=196.29), allyl-3-cyclohexyl propionate (MW=196.29), allyl (cyclohexyloxy)acetate (MW=198.26, Isoananat®), citral diethyl acetal (MW=226.36), benzyl benzoate (MW=212.25), benzyl cinnamate (MW=238.29), 3a,6,6,9a-tetramethyl dodecahydronaphtho[2,1-b]furane (MW=236.40, Ambroxid®), alpha-iron (MW=206.33), beta-iron (MW=206.33), alpha-n-methyl ionone (MW=206.33), beta-n-methyl ionone (MW=206.33), alpha-isomethyl ionone (MW=206.33), beta-isomethyl ionone (MW=206.33) and allyl ionone (MW 232.35), 2,3,8,8-tetramethyl-1,2,3,4,5,6,7,8-octahydro-2-naphthalenyl methyl ketone (MW=234.38), 1-[1,2,3,4,6,7,8,8a-octahydro-1,2,8,8-tetramethyl naphtalen-2-yl]ethanone (MW=234.38), isobornyl acetate (MW=196.29), alpha-ionone (MW=192.30), beta-ionone (MW=192.30), gamma-ionone (MW=192.30), alpha-damascone (MW=192.30), beta-damascone (MW=192.30), delta-damascone (MW=192.30), 1-(2,4,4-trimethyl-2-cyclohexen-1-yl)-2-buten-1-one (MW=192.30, isodamascone), cedrol (MW=222.40), gamma-dodecalactone (MW=198.30), 2-methyl-3-(3,4-methylenedioxyphenyl)propanal (MW=192.22, Helional) and methyl dihydrojasmonate (MW=226.31).

The molecular weights (MW) are shown in brackets and where applicable the brand or product names are given.

The musk fragrance substance(s) of component (B) (ii) are fragrance substances that have a musk odor. These fragrance substances are known to a person skilled in the art since musk is a very important odor direction in perfumery. Musk fragrance substances, including those referred to as preferred in the following, are also described in H. Surburg, J. Panten, “Common Fragrance and Flavor Materials”, 5^{th}. Edition, Wiley-VCH, Weinheim 2006.

Here the musk fragrance substance(s), which are part of component (B) (ii), are preferably selected from the group of macrocyclic musk fragrance substances, polycyclic musk fragrance substances and/or alicyclic musk fragrance substances.

According to the invention preference is for components (B), in particular perfumery fragrance substance mixtures, containing two, three or more different musk fragrance substances as component (B) (ii).

The musk fragrance substance(s) of component (B) (ii) in the context of the present invention are preferably selected from the following Table 1:

TABLE 1

| TYPE | Product name/Brand name | Name/CAS-Name |
|---|---|---|
| MACRO | EXALTENONE | 4-cyclopentadecen-1-one (4Z)-; 4- |
| | | cyclopentadecen-1-one |
| MACRO | CIVETONE | 9-cycloheptadecen-1-one, (9Z)- |
| MACRO | CYCLOHEXADECANOLIDE, | Oxacycloheptadecan-2-one, |
| | DIHYDROAMBRETTOLIDE | ω-hexadecanolide |
| MACRO | ETHYLENE | 1,4-dioxacyclohexadecane-5,16-dione |
| | DODECANDIOATE | |
| MACRO | GLOBALIDE ® | Oxacyclohexadecen-2-one; 15-pentadec- |
| | | (11/12)-enolide |
| MACRO | ETHYLENE BRASSYLATE | 1,4-dioxacycloheptadecane-5,17-dione |
| MACRO | MUSCONE | 3-methy-cyclopentadecanone |
| MACRO | AMBRETTOLIDE | Oxacycloheptadec-10-en-2-one |
| MACRO | MUSCENONE | 3-methyl-cyclopentadecenone |
| MACRO | VELVIONE ®, AMBRETONE | 5-cyclohexadecen-1-one |
| MACRO | AURELIONE ® | 7/8-cyclohexadecen-1-one |
| MACRO | GLOBANONE ® | 8-cyclohexadecen-1-one |
| MACRO | ISOMUSCONE ® | Cyclohexadecanone |
| MACRO | EXALTOLIDE, | Oxacyclohexadecan-2-one |
| | MACROLIDE ® | |
| MACRO | COSMONE ® | 3-methyl-(5E/Z)-cyclotetradecen-1-one |
| POLY | TRASEOLIDE ® | 1-[2,3-dihydro-1,1,2,6-tetramethyl-3-(1- |
| | | methylethyl)-1H-inden-5-yl]-ethanone |
| POLY | PHANTOLIDE ® | 1-(2,3-dihydro-1,1,2,3,3,6-hexamethyl-1H- |
| | | inden-5-yl)-ethanone |
| POLY | TONALIDE ® | 1-(5,6,7,8-tetrahydro-3,5,5,6,8,8- |
| | | hexamethyl-2-naphthalenyl)-ethanon |
| POLY | CRYSOLIDE | 1-[6-(1,1-dimethylethyl)-2,3-dihydro-1,1- |
| | | dimethyl-1H-inden-4-yl]-ethanone |
| POLY | CHROMANOLIDE ® | Tetradecanoic acid, 1-methyl ethyl ester; |
| | | cyclopenta[g]-2-benzopyran, 1,3,4,6,7,8- |
| | | hexahydro-4,6,6,7,8,8-hexamethyl- |
| POLY | GALAXOLIDE ® | Cyclopenta[g]-2-benzopyran, 1,3,4,6,7,8- |
| | | hexahydro-4,6,6,7,8,8-hexamethyl- |
| ALICYC | HELVETOLIDE ® | 1-propanol, 2-[1-(3,3- |
| | | dimethylcyclohexyl)ethoxy]-2-methyl-, 1- |
| | | propanoate |

MACRO = macrocyclic musk fragrance substance
POLY = polycyclic musk fragrance substance
ALICYC = alicyclic musk fragrance substance

Further preference as part of component (B) (ii) is for polycyclic and/or macrocyclic musk fragrance substances, wherein macrocyclic musk fragrance substances in particular have proven to be particularly advantageous for the purpose of the invention, which in turn are preferably selected from the group consisting of macrocyclic C₁₄-C₁₈-ketones and macrocyclic C₁₄-C₁₈-lactones, wherein the ketone or the lactone has a ring size of 15-17 ring atoms and no, one or two oxygen atoms in the ring.

Most preferable are 3-methyl-cyclopentadecenone (Muscenone), 15-pentadec-(11/12)-enolide (Globalide®), ethylene brassylate, oxacyclohexadecan-2-one (Macrolide®), cyclohexadecanone (Isomuscone®), 8-cyclohexadecanone (Globanone®), (7/8)-cyclohexadecanone (Aurelione®) and mixtures thereof.

In a preferred embodiment, component (B) (ii) is selected from the group consisting of 15-pentadec-(11/12)-enolide (Globalide)®, ethylene brassylate and oxacyclohexadecan-2-one (Macrolide®) and mixtures thereof.

The total quantity of the fragrance substance(s) of component (B) (ii) (bottom note) (referred to as preferred in a composition preferred) according to the invention or a cosmetic product preferred according to the invention is 5-80 wt %, particularly preferably 10-70 wt %, in each case in relation to the total weight of component (B), preferably in each case in relation to the total weight of component (B*).

In a composition according to the invention or a cosmetic product according to the invention (in each case in particular in one of the embodiments referred to as preferred) preferably in addition one or more effects are caused, selected from the group of effects consisting of:
reducing the evaporation of fragrance substances, in particular immediately following application of the composition according to the invention and in particular the top note; this also applies to (volatile) fragrance substances, which are normally entrained shortly after application through the evaporation of solvents such as ethanol and water;
stabilizing over time the olfactory profile, that is to say that over a longer period a more constant olfactory profile is achieved;
increasing or stabilizing over time the olfactory impact (the perceived olfactory strength); that is to say that the olfactory impact (the perceived olfactory strength) is not or only insignificantly reduced over a long period;
prolonging the adhesion of the fragrance substances of component (B) of a composition according to the invention, in particular of the volatile fragrance substances (top note), here in turn preferably the fragrance substances of component (B) (i) referred to as preferred, to skin and/or hair in particular to human skin, wherein the retention of component (B) of a composition according to the invention or of a cosmetic product according to the invention is greater compared to an otherwise composition or an otherwise identical product containing no component (A);
imparting a pleasant feeling to the skin of an (ethanolic) fragrance substance composition.

The compositions according to the invention are preferably a component of cosmetic, in particular topical cosmetic, products. In a composition according to the invention or a cosmetic (topical) product according to the invention containing a sensorially (olfactorily) effective quantity of a composition according to the invention, a prolonging of the adhesion (fixing) of the fragrance substances of component (B) is brought about, preferably to skin and/or hair, in particular to human skin. Here preferably one or more further effects described in connection with the present invention are attained, in particular with the embodiments referred to as preferred.

The prolonged adhesion (fixing) of the fragrance substances of component (B) has been observed in particular with volatile fragrance substances (top note) on particular, here in turn preferably the fragrance substances of component (B) (i) referred to as preferred, to skin and/or hair, in particular to human skin.

The retention of component (B) of a composition according to the invention or a cosmetic product according to the invention is preferably at least 40 wt %, preferably at least 50 wt %, more preferably at least 80 wt %, particularly preferably at least 100 wt %, higher compared to an otherwise identical composition or an otherwise identical product containing no component (A), preferably determined as described in Test 1 below.

Cosmetic products and compositions according to the invention are accordingly—in summary—products/compositions with a particularly good adhesion of the top note of a fragrance substance composition, a prolonged and/or intensified impact of the top note, wherein preferably an intensification and/or prolonging of the bottom note is not, or at least not to a sensorially significant extent, observed. All in all, therefore, component (A) discriminates when fixing top and bottom notes in compositions and products according to the invention. The adherence or the perceptibility, in particular of the top note, of fragrance substance compositions, here in particular to human skin, is thereby prolonged and thus an improved olfactory “profile stability” is achieved.

A composition or a (cosmetic) product according to the invention (in particular in one of the embodiments referred to above as preferred) comprises in a preferred embodiment as a further component an effective quantity

(C) of one or more cosmetically acceptable solubilizers for component (B), in particular for component (B*), (B) (i) and/or (B) (ii), preferably selected from the group consisting of

(i) ethanol

and

(ii) dipropylene glycol, diethyl phthalate, triethyl citrate, isopropyl myristate and triacetin, with preference here for dipropylene glycol.

Solubilizers according to component (C), in particular the cosmetically acceptable solubilizers referred to as preferred, are not regarded as fragrance substances in the context of this text, in particular not as perfumery fragrance substances. Component (C), in connection with the present invention, is not to be considered as part of component (B) and accordingly will not be ascribed to it.

Component (C) is advantageous as a solubilizer for component (B), so that a better miscibility and (de)mixing stability of component (B) with component (A) is achieved, thereby allowing easier handling and improved further processing, which is an advantage in the production of cosmetic products according to the invention.

Where a composition according to the invention or a cosmetic product according to the invention contains a solubilizer according to component (C), which is at the same time a fragrance substance, this will, in particular for quantitative considerations, be considered as component (B) and ascribed thereto.

Where a composition according to the invention or a cosmetic product according to the invention contains a solubilizer according to component (C), which is at the same time a diol or triol according to component (D), this will, in particular for quantitative considerations, be considered as component (D) and ascribed thereto.

Where a cosmetic product according to the invention contains component (C) (i) ethanol, the total quantity of component (C) (i) can preferably be up to 95 wt % of ethanol, preferably a maximum of 90 wt %, in each case in relation to the total weight of the product according to the invention.

A composition according to the invention or a cosmetic product according to the invention contains component (C) (ii) preferably in a total quantity of up to 80 wt %, preferably 0.5-60 wt %, preferably 1-50 wt %, more preferably 5-40 wt %, in each case in relation to the total weight of component (B).

A composition according to the invention (in particular in one of the embodiments referred to above as preferred) comprises in an alternative preferred embodiment as a further component

(D) one or more diols or triols with 3-12 C-atoms, preferably selected from the group consisting of glycerin, 1,2-propylene glycol, 1,2-butylene glycol, 1,3-butylene glycol and alkane-diols with 5-12 C-atoms.

Where a composition according to the invention or a cosmetic product according to the invention contains a diol or triol according to component (D), which at the same time is a fragrance substance, this will, in particular for quantitative considerations, be considered as component (D) and ascribed thereto. With component (D), in particular with the diols and triols referred to as preferred, it is not a case of fragrance substances, in particular not a case of perfumery fragrance substances.

Where a composition according to the invention or a cosmetic product according to the invention contains a diol or triol according to component (D), which at the same time is a solubilizer according to component (C), this will, in particular for quantitative considerations, be considered as component (D) and ascribed thereto.

Here the alkane diols with 5-12 C-atoms are preferably selected from the group of straight-chained 1,2-alkane diols with 5-10 C-atoms, in particular from the group consisting of 1,2-pentanediol, 1,2-hexanediol, 1,2-octanediol and/or 1,2-decanediol.

Particularly preferred diols or triols of component (D) are selected from the group consisting of glycerin, 1,2-propylene glycol, 1,2-butylene glycol, 1,3-butylene glycol, 1,2-pentanediol, 1,2-hexanediol, 1,2-octanediol and 1,2-decanediol.

Component (D) can reduce or prevent any undesired turbidity. Additionally, through component (D) the adhesion of a composition according to the invention to (human) skin and/or (human) hair, in particular to human skin, and/or the olfactory profile pattern of a composition according to the invention, can be improved.

A cosmetic preparation according to the invention contains component (D) preferably in a total quantity of 0.2-20 wt %, preferably of 0.5-10 wt %, more preferably of 1-5 wt %, in each case in relation to the total weight of the cosmetic preparation.

In a composition according to the invention (in particular in one of the embodiments referred to above as preferred) component (D) is in particular present in an effective quantity, that is to say in a quantity at which, in the composition, component (D) brings about or intensifies one or more effects, selected from the group of effects consisting of:
further reducing the evaporation of fragrance substances, in particular immediately following application of the composition according to the invention and in particular the top note;
further prolonging the adhesion to (human) skin and/or (human) hair, in particular to human skin (this also means human skin other than the (oral) mucosa), in particular the top note;
an extensively stable olfactory impression following application to the (human) skin over a longer period;
a higher impact following application to the (human) skin;
further stabilizing over time the olfactory profile, that is to say that a more stable olfactory profile over a longer period is achieved;
increasing or further stabilizing over time the olfactory impact (of the perceived olfactory strength); depending on the concentration of component (D) used in each case within the composition according to the invention the impact will be increased over a varying length of time;
prolonging the adhesion of the composition, in particular of the volatile fragrance substances (top note), to skin and/or hair, in particular to human skin;
increasing or prolonging the diffusivity (spatial effect) of the composition, in particular of the volatile fragrance substances (top note), to skin and/or hair, in particular to human skin;
imparting a further improved pleasant feeling to the skin.

A composition according to the invention in particular leaves behind no film on skin and/or hair and is thus not perceived as annoying. A short time after application (i.e. following drying and possible retraction) a composition according to the invention is generally no longer visible (the exception being hair care products such as for example hairspray, with which a film formation is desired).

Other substances that may form part of a (cosmetic) composition according to the invention or a cosmetic (topical) product according to the invention are:

preservatives, preferably those described in US 2006/0089413, abrasives, antiacne and sebum reducing agents, preferably those described in WO 2008/046791, compounds against aging of the skin, preferably those described in WO 2005/123101, antibacterial agents, anti-cellulitis agents, antidandruff agents, preferably those described in WO 2008/046795, antiphlogistic agents, irritation-preventing agents, anti-irritants (anti-inflammatory, irritation-preventing and irritation-inhibiting agents), preferably those described in WO 2007/042472 and US 2006/0089413, antimicrobial agents, preferably those described in WO 2005/123101, antioxidants, preferably those described in WO 2005/123101, astringents, antiseptic agents, antistatics, binders, buffers, support materials, preferably those described in WO 2005/123101, chelating agents, preferably those described in WO 2005/123101, cell stimulants, cleansing agents, conditioning agents, depilators, surface-active substances, deodorizing agents and antiperspirants, preferably those described in WO 2005/123101, softeners, emulsifiers, preferably those described in WO 2005/123101, enzymes, essential oils, preferably those described in US 2008/0070825, insect repellents, preferably those described WO 2005/123101, fibers, film-formers, other fixatives, foaming agents, foam stabilizers, substances to prevent foaming, foam boosters, fungicides, gelling agents and gel forming agents, preferably those described in WO 2005/123101, hair care products, hair styling agents, hair straightening agents (further) moisture regulators (moisture-donating agents, moisturizing and/or moisture-retaining substances), preferably those described in WO 2005/123101, osmolytes, preferably those described in WO 2005/123101, compatible solutes, preferably those described in WO 01/76572 and WO 02/15686, bleaching agents, strengthening agents, stain-removing agents, optically brightening agents, impregnating agents, dirt-repellent agents, friction-reducing agents, lubricants, moisturizing crèmes, ointments, opacifying agents, plasticizing agents, covering agents, polish, gloss agents, polymers, preferably those described in WO 2008/046676, powders, proteins and protein hydrolysates, preferably those described in WO 2005/123101 and WO 2008/046676, (further) lipid replenishing crèmes, polishing agents, skin soothing agents, skin cleansing agents, skin care agents, skin repair agents, preferably containing cholesterol and/or fatty acids and/or ceramides and/or pseudoceramides, preferably those described in WO 2006/053912, skin lightening agents, preferably those described in WO 2007/110415, skin-protecting agents, skin-softening agents, skin-cooling agents, preferably those described in WO 2005/123101, skin-warming agents, preferably those described in WO 2005/123101, stabilizers, UV-absorbing agents and UV-filters, preferably those described in WO 2005/123101, benzylidene-beta-dicarbonyl compounds, preferably those described in WO 2005/107692, alpha-benzoyl-cinnamic acid nitriles, preferably those described in WO 2006/015954, AhR-receptor antagonists, preferably those described in WO 2007/128723 and WO 2007/060256, detergents, fabric conditioners, suspending agents, skin tanning agents, preferably those described in WO 2006/045760, thickening agents, vitamins, preferably those described in WO 2005/123101, oils, waxes and other fats, preferably those described in WO 2005/123101, phospholipids, preferably those described in WO 2005/123101, fatty acids (saturated fatty acids, mono- or polyunsaturated fatty acids, α-hydroxy acids, polyhydroxy-fatty acids), preferably those described in WO 2005/123101, condensers, dyestuffs and color-protecting agents and pigments, preferably those described in WO 2005/123101, anti-corrosive agents, other fragrance substances that are not part of components (B) (i) and (B) (ii), preferably those described in S. Arctander, Perfume and Flavor Chemicals, author's edition, Montclair, N.J., 1969 and Surburg, Panten, Common Fragrance and Flavor Materials, 5th Edition, Wiley-VCH, Weinheim 2006, preferably those explicitly described in US 2008/0070825, alcohols and polyols, preferably those described in WO 2005/123101, surface-active agents, preferably those described in WO 2005/123101, animal extracts, yeast extracts, extracts from algae or microalgae, electrolytes, condensers, organic solvents, preferably those described in WO 2005/123101, or silicones and silicone derivatives preferably those described in WO 2008/046676.

Preferred cosmetic products according to the invention as embodiments of a composition according to the invention are selected from the group consisting of: perfume extracts, eau de parfums, eau de toilettes, after-shaves, eau de colognes, pre-shave products, splash colognes and perfumed refresher tissues and the perfuming of acid, alkaline and neutral cleaning agents, preferably floor cleaners, window glass cleaners, dishwasher detergents, bath and sanitary cleaners, scouring milk, solid and liquid WC cleaners, powdery and foam carpet cleaners, fabric fresheners, ironing aids, liquid washing agents, powder-like washing agents, pre-treatment washing agents such as bleaching agents, soaking agents and stain removers, laundry softeners, washing soaps, washing tablets, disinfectants, surface disinfection agents and air improvers in liquid, gel-like form or in a form applied on a solid carrier, aerosol sprays, waxes and polishes such as furniture polishes, floor waxes, shoe crèmes and body care agents, preferably solid and liquid soaps, shower gels, shampoos, shaving soaps, shaving foams, bath oils, cosmetic emulsions of the oil-in-water, of the water-in-oil and of the water-in-oil-in-water type, preferably skin crèmes and lotions, face crèmes and lotions, day crèmes, night crèmes, sun protection crèmes and lotions, after sun crèmes and lotions, hand crèmes and lotions, foot crèmes and lotions, depilatory crèmes and lotions, after-shave crèmes and lotions, tanning crèmes and lotions, hair care products, preferably hairsprays, hair gels, strengthening hair lotions, hair rinses, permanent and semi-permanent hair dyes, hair shaping means such as cold waves and hair smoothing agents, hair tonics, hair crèmes and lotions, deodorants and antiperspirants, preferably under-arm sprays, roll-ons, deodorant sticks, deodorant crèmes or products of decorative cosmetics, preferably eye shadows, nail varnishes, make ups, lipsticks, mascara as well as candles, lamp oils, joss sticks, insecticides and repellents.

Preferred preparations according to the invention are cosmetic, in particular topical, products, with a conventional composition and are used for cosmetic light protection, for the treatment, care and cleaning of the skin and/or the hair or as a make-up product in decorative cosmetics. Accordingly such preparations take the form of cleaning agents, preferably soap, syndet, liquid washing, shower and bath preparation, skin care agents, preferably emulsion (as a solution, dispersion, suspension; crème, lotion or milk according to the manufacturing method and contents of the oil-in water (O/W) type, the water-in-oil (W/O) type, or multiple emulsion, PIT emulsion, emulsion foam, micro-, nano-emulsion, Pickering emulsion), ointments, pastes, gels (including hydro-, hydrodispersion- and oleogels), alcoholic or aqueous/alcoholic solution, oil, toner, balsam, serum, powder, wipe, eau de toilette, eau de cologne, perfume, wax, including forms of administration such as stick, roll-on, (pump-)spray, aerosol (foaming, non-foaming or after-foaming), skin-care agent (as described above), as a foot-care agent (including keratolytics, deodorants), as insect repellent, as sunscreen, as a self-tanning agent and/or after-sun preparation, skin-care agent as a shaving composition or after-shave, as a depilatory composition, as a hair-care composition, preferably as a shampoo (including as shampoo for normal hair, for greasy hair, for dry, stressed (damaged) hair, shampoo, anti-dandruff shampoo, baby shampoo, shampoo for dry scalp, shampoo concentrate), conditioner, hair tonic, hair lotion, hair rinse, fixature, pomade, permanent wave and setting composition, hair smoothing composition (straightening agent, relaxer), hair setting composition, styling aid (preferably gel or wax); blonding composition, hair dye, preferably temporary, direct pigment, semi-permanent hair dye, permanent hair dye), skin care agents as decorative body-care composition, preferably nail care composition (nail varnish and nail varnish remover), decorative cosmetics (e.g. powder, eye shadow, eye pencil, lipstick), deodorant and/or antiperspirant.

Cosmetic, preferably topical, products according to the invention contain component (A) preferably in a quantity of 0.25-30 wt %, preferably 0.25-20 wt %, more preferably 0.5-15 wt %, particularly preferably 0.5-10 wt %, and quite particularly preferably 1-10 wt %,

and/or

component (B) preferably in a quantity of 0.15-5 wt %, preferably 0.2-3 wt %, more preferably 0.3-3 wt %, particularly preferably 0.3-2.5 wt %, and quite particularly preferably 0.3-2 wt %,

in each case in relation to the total weight of the cosmetic product.

Preferably with the composition according to the invention it is a case of so-called “leave-on” products, i.e. products which remain on (human) skin and/or (human) hair and as a rule are not washed off. Preferably a “leave-on” product according to the invention (in a preferred embodiment of a cosmetic product according to the invention) remains for at least 15 minutes or longer, preferably at least 30 minutes or longer and more preferably at least 60 minutes or longer on the (human) skin and/or the (human) hair. These include in particular eau de parfum, eau de toilette, after-shave, (skin) crèmes, emulsions (for topical application), deodorant sprays, roll-on deodorants, hairsprays and hair conditioners.

Preferred “leave-on” products are selected from the group consisting of:
skin crème or lotion, face crème or lotion, day crème, night crème, sun protection crème, spray or lotion, after-sun crème or lotion, hand crème or lotion, foot crème or lotion, after-shave crème or lotion, skin lightening crème or lotion, skin tanning crème or lotion;
hair care products, preferably hairspray, hair gel, fixing hair lotion, hair tonic, hair crème, hair wax, hair lotion, hair conditioner;
deodorants and antiperspirants, preferably under-arm spray, roll-on, deodorant stick, deodorant crème, and
decorative cosmetic products, preferably eye shadow, nail varnish, lipstick (salves) or mascara.

Preferably a composition according to the invention or a cosmetic product according to the invention is preferably in one of the embodiments referred to above as preferred, comprising:

(A) neopentyl glycol diisononanoate in a total quantity of 0.25-30 wt %, preferably 0.25-20 wt %, more preferably 0.5-15 wt %, particularly preferably 0.5-10 wt %, and quite particularly preferably 1-10 wt %,

and/or

(B) one or more fragrance substances in a quantity of 0.15-5 wt %, preferably 0.2-3 wt %, more preferably 0.3-3 wt %, particularly preferably 0.3-2.5 wt %, and quite particularly preferably 0.3-2 wt %, wherein preferably component (B)
(B) (i) contains 2, 3, 4, 5 or more fragrance substances with a molecular weight in the range of 100 g/mol-175 g/mol, preferably with a molecular weight in the range of 110 g/mol-165 g/mol, preferably in the range of 115 g/mol-160 g/mol, particularly preferably in the range of 120 g/mol-155 g/mol,
and/or
(B) (ii) contains one, 2, 3, 4, 5 or more fragrance substances with a molecular weight of greater than or equal to 190 g/mol (bottom note), preferably in the range of 190 g/mol-300 g/mol, more preferably with a molecular weight in the range of 195 g/mol-290 g/mol, and most preferably in the range of 200-275 g/mol,

and/or

(C) (ii) dipropylene glycol, diethyl phthalate, triethyl citrate, isopropyl myristate and/or triacetin, wherein the total quantity of component (C) (ii) is up to 80 wt %, preferably 0.5-60 wt %, preferably 1-50 wt %, more preferably 5-40 wt %, in each case in relation to the total weight in the range of component (B),

and/or

(D) one or more diols or triols with 3-12 C-atoms, preferably selected from the group consisting of glycerin, 1,2-propylene glycol, 1,2-butylene glycol, 1,3-butylene glycol, 1,2-pentanediol, 1,2-hexanediol, 1,2-octanediol and 1,2-decanediol, in a total quantity of 0.2-20 wt %, preferably of 0.5-10 wt %, more preferably of 1-5 wt %,

in each case in relation to the total weight of the composition or of the product.

The weight percentages given are in each case in relation to the total weight of the composition. Preferably the weight proportions for the components (A), (B), (C) and (D) mentioned as preferred are employed simultaneously.

The present invention also relates to the use of component (A) (as defined above, in particular in an embodiment referred to as preferred above) as an agent for
reducing the evaporation of fragrance substances in a fragrance substance composition, in particular immediately following application;
stabilizing over time the olfactory profile of a fragrance substance composition;
increasing or stabilizing over time the olfactory impact (the perceived olfactory strength) of a fragrance substance composition;
prolonging the adhesion of fragrance substances of a fragrance substance composition, in particular the top note, to skin and/or hair, in particular to human skin;

and/or
imparting or intensifying a pleasant feeling to the skin of an (ethanolic) fragrance substance composition.

The invention also relates to the use of neopentyl glycol diisononanoate as a fixative for perfume oils or as a fixative for (top note) fragrance substances, wherein preferably the top note fragrance substances are selected from the list consisting of:

(B) (i) n-Heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol (2,6-dimethyl-7-octen-2-ol), menthone, isomenthone, 2,6-dimethyl-5-hepten-1-al (Melonal), 3-hexenyl methyl carbonate, benzaldehyde, linalool, tetrahydrolinalool, tetrahydrogeraniol, citral, neral, geranial, benzyl alcohol, benzyl acetate, methyl cinnamate, p-anisaldehyde, menthol, isoamyl acetate, isoamyl butyrate, cis-3-hexenyl acetate, trans-3-hexenyl acetate, hexyl acetate, butyl butyrate, citronellol, nerol, geraniol, 2-phenylethyl alcohol, methyl benzoate, methyl anthranilate, styrallyl acetate, 2,4-dimethyl-3-cyclohexene carboxaldehyde (vertocitral), acetophenone, p-methyl acetophenone, cinammic alcohol, cinammic aldehyde, 3-phenyl propanol (dihydrocinammic alcohol), 2-methyl-1-phenyl-2-propanol (alpha,alpha-dimethyl phenethyl carbinol), anethole, anisalcohol, heliotropin, acetanisole (p-methoxy acetophenone) and agrunitrile (3,7-dimethyl-6-octen-1-nitrile).

The present invention also relates to a method for
reducing the evaporation of fragrance substances in a fragrance substance composition, in particular immediately following application,
stabilizing over time the olfactory profile of a fragrance substance composition;
increasing or stabilizing over time the olfactory impact (the perceived olfactory strength) of a fragrance substance composition,
prolonging the adhesion of fragrance substances of a fragrance substance composition, in particular the top note, to skin and/or hair, in particular to human skin,

and/or
imparting or intensifying a pleasant feeling to the skin of a fragrance substance composition,

with the following step:

mixing of

(A) neopentyl glycol diisononanoate, preferably in a component (B) fixing quantity,

and

(B) one or more fragrance substances, wherein component (B)

(B) (i) contains one, 2, 3, 4, 5 or more fragrance substances with a molecular weight in the range of 100 g/mol-175 g/mol (top note), preferably with a molecular weight in the range of 110 g/mol-165 g/mol, preferably in the range of 115 g/mol-160 g/mol, particularly preferably in the range of 120 g/mol-155 g/mol,

wherein preferably the mass ratio of component (A) to the total mass of component (B) is in the range of 1:20-200:1, preferably in the range of 1:12-100:1, more preferably in the range of 1:6-50:1, particularly preferably in the range of 1:5-33:1, and quite particularly preferably in the range of 1:2-25:1,

and if necessary other components, preferably selected from the group consisting of the components (C) and/or (D) (referred to as preferable above),

wherein the mass ratio relates to the resulting composition after mixing.

The compositions according to the invention can be produced in an easy manner by mixing the individual components of components (A) and (B) and if necessary (C) and/or (D). Here the sequence of contact between the individual components or components is not critical and may be varied.

For the methods and uses according to the invention, that which is stated above concerning preferred compositions and cosmetic (topical) products according to the invention applies by analogy.

The present invention also relates to a method for
imparting, intensifying or modifying an odor on the (human) skin,
prolonging the adhesion of fragrance substances of a fragrance substance composition, in particular the top note, to skin and/or hair, in particular to human skin,

and/or
imparting or intensifying a pleasant feeling to the skin of a fragrance substance composition,

with the following step:
application of a composition according to the invention (preferably a composition referred to above as preferable) or of a (topical) cosmetic product according to the invention (preferably a product referred to above as preferred) to the (human) skin.

The invention also relates to a fragrance substance fixing agent (fixative) kit (combination) comprising

a first component comprising or consisting of

(A) neopentyl glycol diisononanoate,

and

a second component comprising or consisting of

(B) one or more fragrance substances, in particular a fragrance substance mixture comprising 2, 3, 4, 5, 6, 7, 8, 9, 10 or more fragrance substances,

wherein component (B)

(B) (i) contains one, 2, 3, 4, 5 or more fragrance substances with a molecular weight in the range of 100 g/mol-175 g/mol (top note), preferably with a molecular weight in the range of 110 g/mol-165 g/mol, preferably in the range of 115 g/mol-160 g/mol, particularly preferably in the range of 120 g/mol-155 g/mol,

wherein preferably component (B) is selected according to the embodiments referred to above as preferred.

It has also become apparent that component (A) is suitable not only as described above as a fixative for fragrance substances, but also as a solvent for cosmetic UV-light protective filters, in particular for lipophilic (crystalline) UV-light protective filters. Additionally it has been found that component (A), because of the even distribution of the (in particular lipophilic) cosmetic UV-filters, brings about an increase in the SPF, that is to say a higher light protection factor and thus a higher UV protection performance. Here suitable and preferred UV-filters are those mentioned in WO 2005/123101.

On top of this it has been found that component (A) improves the luster of human and animal hair. In this connection, according to one aspect of the present invention, hair-care agents are preferred, preferably selected from the group consisting of shampoo, here in turn preferably shampoo for normal hair, for greasy hair, for dry, stressed (damaged) hair, 2-in-1 shampoo, anti-dandruff shampoo, baby shampoo, shampoo for dry scalp, shampoo concentrate), conditioner, hair tonic, hair lotion, hair rinse, fixature, pomade, permanent wave and setting composition, hair smoothing composition (straightening agent, relaxer), hair setting composition, styling aid (in particular gel or wax); hair dye.

Additionally it has been found that component (A) has a regulating effect on skin moisture, in that the moisture content of (human) skin is improved and the transepidermal water loss (TEWL) is reduced, without having occluding features.

Further Features of Component (A) in Compositions According to the Invention and Cosmetic Products According to the Invention:
high skin absorption
low viscosity at 20° C.
good (cosmetic) moisturizer
good lipid balancing features, e.g. as a component of deodorants, lipsticks or shampoos
very good spraying ability
low polarity
very good solubility in aqueous-alcoholic cosmetic products
provides a water-repellent effect
highly resistant to oxidation
protects the (human) skin from drying out
non-occlusive
gives (human) hair luster, elasticity and softness
provides a pleasant, soft, smooth and sleek feeling to the skin.

Due to the stated features of component (A) in compositions according to the invention and cosmetic products according to the invention, the following types of formulation and areas of application are particularly preferred:
emulsions, in particular due to their exceptional spraying ability,
hair care products, in particular because they give hair luster, elasticity and softness,
soaps, because they counteract the drying out of (human) skin and reduce or prevent drying out,
deodorants, antiperspirants, lip salves and lipsticks, since they are moisturizing and skin-friendly,
ethanolic or aqueous-ethanolic cosmetic products, such as ethanolic fragrance substance mixtures, eau de toilettes or deodorants, since they reduce or prevent skin irritation caused by ethanol.

The following examples explain the invention; unless otherwise stated all proportions and percentages refer to the weight.

### EXAMPLES

Abbreviations used: SPF=sun protection factor; MW=molecular weight; Ret.=retention increase; DPG=dipropylene glycol

Test 1: Investigation of the Reduced Release of Fragrance Substances from a Fragrance Substance Mixture Containing One Mass Equivalent of Neopentyl Glycol Diisononanoate

Test Method:

50 μl of a fragrance substance mixture containing the fragrance substances listed in the following table were dissolved to 0.5% in EtOH. One aliquot of the ethanolic solution was added to an equal quantity of neopentyl glycol diisononanoate, i.e. the weight ratio of the total mass of fragrance substances and neopentyl glycol diisononanoate was 1:1.

Each of the two solutions was applied to a defined area of fragrance substance testing paper (2 cm²) and equilibrated for 15 minutes at 22° C. Then the fragrance substance testing papers were each extracted with 4 ml acetone and diluted with 100 μg diphenyl methane as the internal standard (IS). The samples were then measured using GC/MS, and the remaining amounts of fragrance substances remaining on the fragrance substance testing papers were quantified using the IS method.

The following table shows the results of the quantitative analyses performed and the percentage increase in retention calculated from these for each of the perfumery fragrance substances.

| Fragrance | | GC-Counts | GC-Counts | | | |
|---|---|---|---|---|---|---|
| substance | MW | with (A) | without (A) | Ret. % | Component | |
| n-heptanol | 118.23 | 233939 | 24436 | 857 | (B) (i) | |
| 2-phenyl ethyl alcohol | 122.17 | 2061675 | 1368972 | 51 | (B) (i) | |
| Anisaldehyde | 136.15 | 3073838 | 2145579 | 43 | (B) (i) | |
| Agrunitrile | 151.25 | 2069626 | 877864 | 136 | (B) (i) | |
| Camphor | 152.24 | 239895 | 27060 | 787 | (B) (i) | |
| Geraniol | 154.25 | 2278603 | 1654064 | 138 | (B) (i) | |
| Linalool | 154.25 | 676146 | 106166 | 537 | (B) (i) | |
| Dihydromyrcenol | 156.27 | 609603 | 72942 | 736 | (B) (i) | |
| γ-decalactone | 170.25 | 3948156 | 3576703 | 10 | (B) (i) | |
| β-ionone | 192.30 | 4447058 | 3543135 | 26 | | (B) (ii) |
| Lilial ® | 204.31 | 5471716 | 4864188 | 13 | | (B) (ii) |
| Benzyl benzoate | 212.25 | 6493182 | 6405967 | 1 | | (B) (ii) |
| Hexyl salicylate | 222.28 | 4395718 | 4006701 | 10 | | (B) (ii) |
| Cedrol | 222.40 | 6647973 | 5706666 | 16 | | (B) (ii) |
| Macrolide ® | 240.38 | 6052651 | 5872871 | 3 | | (B) (ii) |

Agrunitrile = 3,7-dimethyl-6-octen-1-nitrile; Lilial ® = 2-methyl-3-(4-tert-butyl phenyl)propanal; Macrolide ® = oxacyclohexadecan-2-one.

The results of the quantitative investigations clearly demonstrate the fixing features of neopentyl glycol diisononanoate. They clearly show that in the presence of neopentyl glycol diisononanoate the evaporation rate of fragrance substances, in particular of top note fragrance substances, can be significantly reduced, clearly demonstrating the fixing features of this ester for fragrance substances. These analytical results were able to be reproduced sensorially.

| | | wt % | | | | | | | | | | | |
|---|---|---|---|---|---|---|---|---|---|---|---|---|---|
| Material | INCI Name | 1 | 2 | 3 | 4 | 5 | 6 | 7 | 8 | 9 | 10 | 11 | 12 |
| Neopentyl glycol | 2,2-dimethyl-1,3- | 0.5 | 0.3 | 10.0 | 1.0 | 6.0 | 5.0 | 2.5 | 7.0 | 2.0 | 6.0 | 4.5 | 7.0 |
| diisononanoate | propandiol | | | | | | | | | | | | |
| | diisononaoate | | | | | | | | | | | | |
| (−) Alpha bisabolol, | Bisabolol | 0.1 | | | | | | | | | 0.2 | | 0.3 |
| natural | | | | | | | | | | | | | |
| Abil 350 | Dimethicone | | | | | | | | 2.0 | | | | |
| Aluminum stearate | Aluminum Stearate | | | | | | | | | | | | 1.2 |
| Avocado oil | Persea Gratissima | | | | | | | | | | | 3.0 | |
| | (Avocado) Oil | | | | | | | | | | | | |
| L-arginine | Arginine | | | | | | | | | 0.5 | | | |
| Carbopol Ultrez-10 | Carbomer | | | | | | | | 0.2 | | 0.2 | | |
| Cosmetic color, brown, | Color | | | | | | | | | | | 4.0 | |
| powder | | | | | | | | | | | | | |
| Covi-Ox T-70 | Tocopherol | | | 0.1 | | | | | | | | 0.1 | |
| Cutina GMS V | Glyceryl Stearate | | | | | | | | 2.0 | | 2.0 | | |
| DC 9701 powder | Dimethicone/Vinyl | | | | | | | | | | | | |
| | Dimethicone | | | | | | | | | | | | |
| | Crosspolymer | | | | | | | | | | | | |
| Dehydol LS3 Deo | Laureth-3 | | | | | | 8.0 | | | | | | |
| Deolite | Dimethyl | | | | | | | 0.2 | | | | | |
| | Phenylpropanol | | | | | | | | | | | | |
| | Pentylene Glycol | | | | | | | | | | | | |
| Diammonium citrate | Diammonium Citrate | | | | | | | | | | | | |
| Dow Corning 246 fluid | Cyclohexasiloxane | | | | | | | 1.0 | | | | 2.0 | |
| Dow Corning 345 fluid | Cyclomethicone | | | | | | | | | | | | |
| D-Panthenol 75 L | Panthenol | | | | | | | | | | | 1.0 | |
| Dracorin ® 100 S.E.P. | Glyceryl Stearate, | | | | | | | 0.5 | | | | | |
| | PEG-100 Stearate | | | | | | | | | | | | |
| Dracorin ® CE | Glyceryl | | | | | | | | | 2.0 | | | |
| | Stearate/Citrate | | | | | | | | | | | | |
| Dracorin ® GOC | Glyceryl Oleate Citrate | | | | | | | 2.0 | | | | 2.0 | |
| | Caprylic Capric | | | | | | | | | | | | |
| | Triglyceride | | | | | | | | | | | | |
| Drago-Beta-Glucan | Water (Aqua), | | | | | | | | | | 2.0 | | |
| | Butylene Glycol, | | | | | | | | | | | | |
| | Glycerin, Avena Sativa | | | | | | | | | | | | |
| | (Oat) Kernel Extract | | | | | | | | | | | | |
| DragoCalm ® | Water, Glycerin, | | | | | | | | | | 1.0 | | |
| | Avena Sativa (Oat | | | | | | | | | | | | |
| | Kernel Extract) | | | | | | | | | | | | |
| Dragocide ® Liquid | Phenoxyethanol, | | | | | | | 0.8 | 0.8 | | | | 0.8 |
| | Methylparaben, | | | | | | | | | | | | |
| | Ethylparaben, | | | | | | | | | | | | |
| | Butylparaben, | | | | | | | | | | | | |
| | Propylparaben, | | | | | | | | | | | | |
| | Isobutylparaben | | | | | | | | | | | | |
| Dragoderm ® | Glycerin, Triticum | | | | | | | | | | 2.0 | 2.0 | |
| | Vulgare (Wheat) | | | | | | | | | | | | |
| | Gluten, Water (Aqua) | | | | | | | | | | | | |
| Dragosan W/O P | Sorbitan Isostearate, | | | | | | | | | | | | 8.0 |
| | Hydrogenated Castor | | | | | | | | | | | | |
| | Oil, Ceresin, Beeswax | | | | | | | | | | | | |
| | (Cera Alba) | | | | | | | | | | | | |
| Dragosantol ® 100 | Bisabolol | | | 0.2 | | 0.3 | | 0.2 | | | | | |
| Dragosine ® | Carnosine | | | | | | | | | | 0.2 | | |
| Dragoxat ® 89 | Ethylhexyl | | | 15.0 | | 4.0 | | | | 3.0 | 4.0 | | 5.0 |
| | Isononanoate | | | | | | | | | | | | |
| EDTA | Disodium EDTA | | | | | | | | | 0.1 | 0.1 | | |
| Emulsiphos ® | Potassium Cetyl | | | | | | | | 2.0 | | 2.0 | | |
| | Phosphate, | | | | | | | | | | | | |
| | Hydrogenated Palm | | | | | | | | | | | | |
| | Glycerides | | | | | | | | | | | | |
| Ethanol | Ethanol | 28.0 | 45.2 | | To | | | | 5.0 | | | 5.0 | |
| | | | | | 100 | | | | | | | | |
| Color, brown, E 172 + | Color | | | | | | | | | | 4.0 | | |
| E 171 | | | | | | | | | | | | | |
| Perfume oil “P1” | Perfume | 1.0 | 0.3 | 0.3 | 10.0 | 0.5 | 2.0 | | | | | | |
| Perfume oil “P2” | Perfume | | | | | | | 0.4 | 0.3 | 0.4 | 0.3 | 0.5 | 0.3 |
| Frescolat ® ML | Menthyl Lactate | | | | | | | 0.5 | 0.3 | | | 0.5 | |
| Fruitapone ® Orange B | Propylene Glycol, | | | | | | | | 1.0 | | | | |
| | Water (Aqua), Citric | | | | | | | | | | | | |
| | Acid, Citrus Aurantium | | | | | | | | | | | | |
| | Dulcis (Orange) Juice, | | | | | | | | | | | | |
| | Trideceth-9, Bisabolol | | | | | | | | | | | | |
| Glycerin 85% in water | Glycerin | | | | | | | | 2.0 | | 3.0 | | 3.0 |
| Glycerin | Glycerin | | | | | | | 4.0 | | | | 4.0 | |
| Hydrolite ®-5 | Pentylene Glycol (1,2- | | | | | | | | | 5.0 | | 5.0 | |
| | Pentanediol) | | | | | | | | | | | | |
| Hydroviton ®-24 | Water, Pentylene | | | | | | | | | | 1.0 | | 2.0 |
| | Glycol, Glycerin, Lactic | | | | | | | | | | | | |
| | Acid, Sodium Lactate, | | | | | | | | | | | | |
| | Serine, Urea, Sorbitol, | | | | | | | | | | | | |
| | Sodium Chloride, | | | | | | | | | | | | |
| | Allantoin | | | | | | | | | | | | |
| Iso Adipat | Diisopropyl Adipate | | | | | | | | | | 1.0 | | |
| Isodragol ® | Triisononanoin | | | | | | | 1.0 | | | | | |
| Jojoba oil | Simmondsia Chinensis | | | | | | | | | | | | 2.0 |
| | (Jojoba) Seed Oil | | | | | | | | | | | | |
| Keltrol CG RD | Xanthan Gum | | | | | | | | 0.1 | 0.1 | 0.2 | | |
| Lanette O | Cetearyl Alcohol | | | | | | | | 3.0 | 2.0 | 3.0 | | |
| Luviskol K30 | PVP | | 4.5 | | | | | | | | | | |
| Mineral oil | Mineral Oil | | | | | 64.2 | | | | | | | 8.0 |
| Neo Heliopan ® 303 | Octocrylene | | | | | | | | | 5.0 | | | |
| Neo Heliopan ® 357 | Butylmethoxydibenzoyl | | | | | 1.5 | | | | 4.0 | 1.5 | | |
| | methane | | | | | | | | | | | | |
| Neo Heliopan ® E 1000 | Isoamyl | | | | | | | | | | 5.0 | | |
| | p•Methoxycinnamate | | | | | | | | | | | | |
| Neo Heliopan ® Hydro, | Phenylbenzimidazole | | | | | | | | | 8.0 | 3.3 | | |
| 25% in water, | Sulfonic Acid | | | | | | | | | | | | |
| neutralized with L- | | | | | | | | | | | | | |
| arginine | | | | | | | | | | | | | |
| Neo Heliopan ® MBC | 4-Methylbenzylidene | | | | | 1.5 | | | | | | | |
| | Camphor | | | | | | | | | | | | |
| Neo Heliopan ® OS | Ethylhexyl Salicylate | | | | | | | | | 5.0 | | | |
| Neutral oil | Caprylic/Capric | | | To | | 22.0 | To | 3.5 | | | | 5.0 | |
| | Triglyceride | | | 100 | | | 100 | | | | | | |
| Ozokerite wax 2389 | Ozokerite | | | | | | | | | | | | 2.0 |
| Pemulen TR-2 | Acrylates/C10-30 Alkyl | | | | | | | 0.3 | | | | 0.3 | |
| | Acrylate Crosspolymer | | | | | | | | | | | | |
| Polyglycol 400 | PEG-8 | | 0.1 | | | | | | | | | | |
| Potassium sorbate | Potassium Sorbate | | | | | | | | | | | 0.1 | |
| Propane, butane 2,7 | Propane, Butane | To | To | | | | | | | | | | |
| bar | | 100 | 100 | | | | | | | | | | |
| Propylene glycol | Propylene Glycol | | | | | | | | 3.0 | 4.0 | | | |
| Rezal 36 GP | Aluminum Zirconium | | | | | | | 5.0 | | | | | |
| | Tetrachlorohydrex | | | | | | | | | | | | |
| | GLY | | | | | | | | | | | | |
| Sodium chloride | Sodium Chloride | | | | | | | | | | | | 1.0 |
| NaOH, 10% in water | Sodium Hydroxide | | | | | | | 0.6 | 0.5 | | | | 0.4 |
| Softisan 100 | Hydrogenated Coco | | | | | | | | | 1.5 | | | |
| | Glycerides | | | | | | | | | | | | |
| Solubilizer | PEG-40Hydrogenated | | | | 1.0 | | | | | | | | |
| | Castor Oil, Trideceth- | | | | | | | | | | | | |
| | 9, Propylene Glycol, | | | | | | | | | | | | |
| | Water (Aqua) | | | | | | | | | | | | |
| Squalane, vegetable | Squalane | | | | | | | | | | 3.0 | | |
| Almond oil | Prunus Dulcis | | | 20.0 | | | | | | | | | |
| SymCalmin ® | Pentylene Glycol, | | | | | | | | | | | | |
| | Butylene Glycol, | | | | | | | | | | | | |
| | Hydroxyphenyl | | | | | | | | | | | | |
| | Propamidobenzoic | | | | | | | | | | | | |
| | Acid | | | | | | | | | | | | |
| SymClariol ® | Decylene Glycol | 0.5 | | | | | | | | | | | |
| SymDeo ® MPP | Dimethyl | 0.5 | | | | | | | | | | | |
| | Phenylbutanol | | | | | | | | | | | | |
| SymDiol ® 68 | 1,2 Hexanediol, | | | | | | | | | | 1.0 | | |
| | Caprylyl Glycol (1:1) | | | | | | | | | | | | |
| SymGlucan ® | Water (Aqua) Glycerin, | | | | | | | | | | | 1.0 | |
| | Beta Glucan | | | | | | | | | | | | |
| SymMollient ® W/S | Trideceth-9, PEG-5 | | | | 1.0 | | | | | | | | |
| | Isononanoate | | | | | | | | | | | | |
| SymRelief ® | Bisabolol, Zingiber | | | | | | | | | | | 0.2 | |
| | Officinale (Ginger) | | | | | | | | | | | | |
| | Root Extract | | | | | | | | | | | | |
| SymRepair ® | Hexyldecanol, | | | | | | | | | | 2.0 | 3.0 | |
| | Bisabolol, | | | | | | | | | | | | |
| | Cetylhydroxyproline | | | | | | | | | | | | |
| | Palmitamide, Stearic | | | | | | | | | | | | |
| | Acid, Brassica | | | | | | | | | | | | |
| | Campestris (Rapeseed | | | | | | | | | | | | |
| | Sterols) | | | | | | | | | | | | |
| SymVital ™ | Aloe Barbadensis Leaf | | | | | | | | 0.1 | | | | |
| | Juice Powder, | | | | | | | | | | | | |
| | Magnesium Ascorbyl | | | | | | | | | | | | |
| | Phosphate, Rubus | | | | | | | | | | | | |
| | Idaeus (Raspberry) | | | | | | | | | | | | |
| | Leaf Extract | | | | | | | | | | | | |
| Triethanolamine | Triethanolamine | | | | | | | | | | 0.9 | 0.3 | |
| Vitamin-E-acetate | Tocopherol Acetate | | | 0.5 | | | | | | 0.5 | | | 0.2 |
| Water | Water (Aqua) | | | | 8.0 | | | To | To | To | To | To | To |
| | | | | | | | | 100 | 100 | 100 | 100 | 100 | 100 |

The perfume oil “P1” used in formulation examples 1-6 with a rose odor had the following composition:

| | Parts by |
|---|---|
| Component/NAME | weight |
| Acetophenone, 10% in DPG | 10.00 |
| n-undecanal | 5.00 |
| Gamma-undecalactone | 15.00 |
| Allyl amyl glycolate, 10% in DPG | 20.00 |
| Amyl salicylate | 25.00 |
| Benzyl acetate | 60.00 |
| Citronellol | 80.00 |
| D-limonene | 50.00 |
| Trans-9-decen-1-ol | 15.00 |
| Dihydromyrcenol | 50.00 |
| Dimethyl benzyl carbinyl acetate | 30.00 |
| Diphenyl oxide | 5.00 |
| GALAXOLIDE ® | 20.00 |
| Geraniol | 40.00 |
| Nerol | 20.00 |
| Geranium oil | 15.00 |
| Hexenol cis-3, 10% in DPG | 5.00 |
| Hexenyl salicylate cis-3 | 20.00 |
| Indol, 10% in DPG | 10.00 |
| Alpha-ionone | 15.00 |
| Beta-ionone | 5.00 |
| Lilial ® (2-methyl-3-(4-tert-butyl-phenyl)propanal) | 60.00 |
| Linalool | 40.00 |
| Methyl phenyl acetate | 10.00 |
| Phenyl ethyl alcohol | 245.00 |
| Styrolyl acetate | 20.00 |
| Terpineol | 30.00 |
| Tetrahydrolinalool | 50.00 |
| Ethylene brassylate | 30.00 |
| Total: | 1,000.00 |

The perfume oil “P2” used in formulation examples 7-12 with a white flowery fragrance substance and musk note had the following composition:

| | Parts by |
|---|---|
| Component/NAME | weight |
| Benzyl acetate | 60.00 |
| Citronellyl acetate | 60.00 |
| Cyclamen aldehyde (2-methyl-3-(4-isopropylphenyl)propanal | 20.00 |
| Dipropylene glycol | 60.00 |
| Ethyl linalool | 40.00 |
| Florol (2-isobutyl-4-methyltetrahydro-2H-pyran-4-ol) | 30.00 |
| Globanone [(E/Z)-8-cyclohexadecen-1-one] | 100.00 |
| Ethylene brassylate | 80.00 |
| Hedione ® (methyl dihydrojasmonate) | 140.00 |
| Hexenyl salicylate, cis-3 | 10.00 |
| Vertocitral (2,4-dimethyl-3-cyclohexene carboxaldehyde) | 5.00 |
| Hydratropa aldehyde, 10% in DPG | 5.00 |
| Isodamascone (1-(2,4,4-trimethyl-2-cyclohexen-1-yl)-2-buten- | 5.00 |
| 1-one), 10% in DPG | |
| Cyclohexadecanone | 40.00 |
| Jacinthaflor (2-methyl-4-phenyl-1,3-dioxolan) | 10.00 |
| Cis-jasmone, 10% in DPG | 20.00 |
| Linalool | 50.00 |
| Linalyl acetate | 30.00 |
| Methyl benzoate, 10% in DPG | 25.00 |
| Para-methyl cresol, 10% in DPG | 10.00 |
| Nerol | 20.00 |
| Phenyl propyl aldehyde | 5.00 |
| 2-phenyl ethyl alcohol | 82.00 |
| Tetrahydrogeraniol | 13.00 |
| 2,2-dimethyl-3-cyclohexyl-1-propanol | 40.00 |
| Tonalide ® | 40.00 |
| Total: | 1,000.00 |

1. A composition comprising
(A) neopentyl glycol diisononanoate,
and
(B) one or more fragrance substances,
wherein component (B) comprises
(B)(i) one or more fragrance substances with a molecular weight in a range of 100 g/mol-175 g/mol (top note).

2. The composition as claimed in claim 1, wherein a mass ratio of component (A) to a total mass of component (B) is in a range of 1:20-200:1.

3. The composition as claimed in claim 1, wherein component (B) further comprises
(B) (ii) one or more fragrance substances with a molecular weight in a range of greater than or equal to 190 g/mol (bottom note).

4. The composition as claimed in claim 1, wherein the one or more fragrance substances of component (B)(i) (top note) are selected from the group consisting of:
(B)(i) n-Heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol(2,6-dimethyl-7-octen-2-ol), menthone, isomenthone, 2,6-dimethyl-5-hepten-1-al (Melonal), 3-hexenyl methyl carbonate, benzaldehyde, linalool, tetrahydrolinalool, tetrahydrogeraniol, citral, neral, geranial, benzyl alcohol, benzyl acetate, methyl cinnamate, p-anisaldehyde, menthol, isoamyl acetate, isoamyl butyrate, cis-3-hexenyl acetate, trans-3-hexenyl acetate, hexyl acetate, butyl butyrate, citronellol, nerol, geraniol, 2-phenylethyl alcohol, methyl benzoate, methyl anthranilate, styrallyl acetate, 2,4-dimethyl-3-cyclohexene carboxaldehyde (vertocitral), acetophenone, p-methyl acetophenone, cinammic alcohol, cinammic aldehyde, 3-phenyl propanol (dihydrocinammic alcohol), 2-methyl-1-phenyl-2-propanol (alpha,alpha-dimethyl phenethyl carbinol), anethole, anisalcohol, heliotropin, acetanisole (p-methoxy acetophenone) and agrunitrile (3,7-dimethyl-6-octen-1-nitrile).

5. The composition as claimed in claim 3, wherein the one or more fragrance substances of component (B)(ii) are selected from the group consisting of:
(B)(ii) a musk fragrance substance, alpha-n-amylcinnamaldeyde (MW=202.30), alpha-iso-amylcinnamaldehyde (MW=202.30), alpha-n-hexylcinnamaldehyde (MW=216.32), alpha-iso-hexylcinnamaldehyde (MW=216.32), benzyl salicylate (MW=228.25), cis-3-hexenyl salicylate (MW=220.27), isoamyl salicylate (MW=208.26), hexyl salicylate (MW=222.28), 2-methyl-3-(4-tert-butylphenyl)propanal (MW=204.31; Lilial®), 2-methyl-3-(4-isopropylphenyl)propanal (MW=190.28, cyclamen aldehyde), 2,3,8,8-tetramethyl-1,2,3,4,5,6,7,8-octahydro-2-naphthalenyl methyl ketone (MW=234.38, Iso E Super®), 1-[1,2,3,4,6,7,8,8a-octahydro-1,2,8,8-tetramethyl naphtalen-2-yl]ethanone (MW=234.38, Iso E Super®), methyl dihydrojasmonate (MW=226.32, Hedione®), linalyl acetate (MW=196.29), ethyllinalyl acetate (MW=210.31), nerolidol (MW=222.37), farnesol (MW=222.37), cedryl methyl ether (MW=236.40, Cedramber), cedryl methyl ketone (MW=246.39), cedryl acetate (MW=264.41), (4aR,5R,7aS,9R)-octahydro-2,2,5,8,8,9a-hexamethyl-4H-4-a,9-methanoazuleno(5,6-d) 1,3-dioxol) (MW=278.44, Ambrocenide®), hexahydro-1′,1′,5′,5′-tetramethyl-spiro[1,3-dioxolan,2,8′(5′H)-[2H-2,4-a]-methanonaphthalene (MW=264.41 ethylenedioxy-3H-isolongifolane, Ysamber® K), 2-methyl-4-(2,2,3-trimethyl-3-cyclopenten-1-yl)butanol (MW=196.34, Brahmanol), 5-(2,2,3-trimethyl-3-cyclopenten-1-yl)-3-methylpentan-2-ol (MW=210.36, Sandalore®), 2-ethyl-4-(2,2,3-trimethyl-3-cyclopenten-1-yl)-2-buten-1-ol (MW=208.35, Sandranol®), 3-methyl-5-(2,2,3-trimethyl-3-cyclopenten-1-yl)-4-penten-2-ol (MW=208.35, Ebanol®), 3,3-dimethyl-5-(2,2,3-trimethyl-3-cyclopenten-1-yl)-4-penten-2-ol (MW=222.37, Polysantol®), 3-isocamphylcyclohexanol (MW=236.40, Sandel 80®), 1-(2,2,6-trimethylcyclohexyl)hexan-3-ol (MW=226.41, Timberol®), cyclododecyl methyl ether (MW=198.35, Palisandin), (ethoxymethoxy)cyclododecane (MW=242.41, Boisambrene Forte®), 1-methyl-4-(4-methyl-3-penten-1-yl)-3-cyclohexene carboxaldehyde (MW=206.33, Precyclemone B®), 4-(4-hydroxy-4-methylpentyl)-3-cyclohexene carboxaldehyde (MW=210.32, Lyral®), 2-methyl-4-(2,2,6-trimethyl-1-cyclohexen-1-yl)-2-butenal (MW=206.33, Boronal), decahydro-beta-naphthyl acetate (MW=196.29), allyl-3-cyclohexyl propionate (MW=196.29), allyl (cyclohexyloxy)acetate (MW=198.26, Isoananat®), citral diethyl acetal (MW=226.36), benzyl benzoate (MW=212.25), benzyl cinnamate (MW=238.29), 3a,6,6,9a-tetramethyl dodecahydronaphtho[2,1-b]furane (MW=236.40, Ambroxid®), alpha-iron (MW=206.33), beta-iron (MW=206.33), alpha-n-methyl ionone (MW=206.33), beta-n-methyl ionone (MW=206.33), alpha-isomethyl ionone (MW=206.33), beta-isomethyl ionone (MW=206.33) and allyl ionone (MW 232.35), 2,3,8,8-tetramethyl-1,2,3,4,5,6,7,8-octahydro-2-naphthalenyl methyl ketone (MW=234.38), 1-[1,2,3,4,6,7,8,8a-octahydro-1,2,8,8-tetramethyl naphtalen-2-yl]ethanone (MW=234.38), isobornyl acetate (MW=196.29), alpha-ionone (MW=192.30), beta-ionone (MW=192.30), gamma-ionone (MW=192.30), alpha-damascone (MW=192.30), beta-damascone (MW=192.30), delta-damascone (MW=192.30), 1-(2,4,4-trimethyl-2-cyclohexen-1-yl)-2-buten-1-one (MW=192.30, isodamascone), cedrol (MW=222.40), gamma-dodecalactone (MW=198.30), 2-methyl-3-(3,4-methylenedioxyphenyl)propanal (MW=192.22, Helional) and methyl dihydrojasmonate (MW=226.31).

6. The composition as claimed in claim 1, further comprising
(C) one or more cosmetically acceptable solubilizers for component (B) selected from the group consisting of
(i) ethanol
and
(ii) dipropylene glycol, diethyl phthalate, triethyl citrate, isopropyl-myristate and triacetin.

7. The composition as claimed in claim 1, further comprising
(D) one or more diols or triols with 3-12 C-atoms.

8. A topical cosmetic product comprising a sensorially effective quantity of
a) a composition as defined in claim 1.

9. The composition as claimed in claim 1 comprising
(A) neopentyl glycol diisononanoate in a total quantity of 0.25-30 wt. %
and
(B) one or more fragrance substances in a quantity of 0.15-5 wt. %, wherein component (B) comprises
(B)(i) one or more fragrance substances with a molecular weight in the range of 100 g/mol-175 g/mol (top note),
and/or
(B)(ii) one or more fragrance substances with a molecular weight in a range of greater than or equal to 190 g/mol (bottom note),
and/or
(C) one or more cosmetically acceptable solubilizers for component (B) selected from the group consisting of
(C)(ii) dipropylene glycol, diethyl phthalate, triethyl citrate, isopropyl myristate and triacetin, wherein the total quantity of component (C) (ii) is up to 80 wt % in relation to a total weight of component (B),
and/or
(D) one or more diols or triols with 3-12 C-atoms in a total quantity of 0.2-20 wt. %
in relation to a total weight of the composition.

10. A method for
reducing the evaporation of fragrance substances in a fragrance substance composition,
stabilizing over time the olfactory profile of a fragrance substance composition,
increasing or stabilizing over time the olfactory impact (the perceived olfactory strength) of a fragrance substance composition,
prolonging the adhesion of fragrance substances of a fragrance substance composition to skin and/or hair
and/or
imparting or intensifying a pleasant feeling to the skin
comprising mixing
(A) neopentyl glycol diisononanoate and
(B) one or more fragrance substances, wherein component (B) comprises
(B)(i) one or one or more fragrance substances with a molecular weight in a range of 100 g/mol-175 g/mol (top note).

11. A method for
imparting, intensifying or modifying an odor on human skin,
prolonging the adhesion of fragrance substances of a fragrance substance composition to human skin and/or human hair and/or
imparting or intensifying a pleasant feeling to human skin
comprising applying a composition as claimed in claim 1 to human skin and/or human hair.

12. The method as claimed in claim 10, wherein the fragrance substance composition comprises one or more fragrance substances selected from the group consisting of:
(B)(i) n-heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol(2,6-dimethyl-7-octen-2-ol), menthone, isomenthone, 2,6-dimethyl-5-hepten-1-al (Melonal), 3-hexenyl methyl carbonate, benzaldehyde, linalool, tetrahydrolinalool, tetrahydrogeraniol, citral, neral, geranial, benzyl alcohol, benzyl acetate, methyl cinnamate, p-anisaldehyde, menthol, isoamyl acetate, isoamyl butyrate, cis-3-hexenyl acetate, trans-3-hexenyl acetate, hexyl acetate, butyl butyrate, citronellol, nerol, geraniol, 2-phenylethyl alcohol, methyl benzoate, methyl anthranilate, styrallyl acetate, 2,4-dimethyl-3-cyclohexene carboxaldehyde (vertocitral), acetophenone, p-methyl acetophenone, cinammic alcohol, cinammic aldehyde, 3-phenyl propanol (dihydrocinammic alcohol), 2-methyl-1-phenyl-2-propanol (alpha,alpha-dimethyl phenethyl carbinol), anethole, anisalcohol, heliotropin, acetanisole (p-methoxy acetophenone) and agrunitrile (3,7-dimethyl-6-octen-1-nitrile).

13. A method of fixing a fragrance (top note) to skin or hair comprising applying to the skin or hair a composition comprising a mixture of
(A) neopentyl glycol diisononanoate,
and
(B) one or more fragrance substances, wherein component (B) comprises
one or more top note fragrance substances selected from the group consisting of:
(B)(i) n-heptanol, trans-9-decen-1-ol, camphor, alpha-pinene, beta-pinene, gamma-terpinene, alpha-phellandrene, alpha-terpineol, borneol, limonene, 6-methyl-5-hepten-2-one, n-octanal, eucalyptol (1,8-cineol), rose oxide, 3-hexenol, dihydromyrcenol(2,6-dimethyl-7-octen-2-ol), menthone, isomenthone, 2,6-dimethyl-5-hepten-1-al (Melonal), 3-hexenyl methyl carbonate, benzaldehyde, linalool, tetrahydrolinalool, tetrahydrogeraniol, citral, neral, geranial, benzyl alcohol, benzyl acetate, methyl cinnamate, p-anisaldehyde, menthol, isoamyl acetate, isoamyl butyrate, cis-3-hexenyl acetate, trans-3-hexenyl acetate, hexyl acetate, butyl butyrate, citronellol, nerol, geraniol, 2-phenylethyl alcohol, methyl benzoate, methyl anthranilate, styrallyl acetate, 2,4-dimethyl-3-cyclohexene carboxaldehyde (vertocitral), acetophenone, p-methyl acetophenone, cinammic alcohol, cinammic aldehyde, 3-phenyl propanol (dihydrocinammic alcohol), 2-methyl-1-phenyl-2-propanol (alpha,alpha-dimethyl phenethyl carbinol), anethole, anisalcohol, heliotropin, acetanisole (p-methoxy acetophenone) and agrunitrile (3,7-dimethyl-6-octen-1-nitrile).

14. A method for
reducing the evaporation of fragrance substances in a fragrance substance composition;
stabilizing over time the olfactory profile of a fragrance substance composition;
increasing or stabilizing over time the olfactory impact (the perceived olfactory strength) of a fragrance substance composition;
prolonging the adhesion of fragrance substances of a fragrance substance composition to skin and/or hair, in particular to human skin;
and/or
imparting or intensifying a pleasant feeling to the skin of a fragrance substance composition
comprising combining the fragrance substance composition with neopentyl glycol diisononanoate.

15. A fragrance substance fixative kit comprising
(A) neopentyl glycol diisononanoate,
and
(B) one or more fragrance substances,
wherein component (B) comprises
(B)(i) one or more fragrance substances with a molecular weight in the range of 100 g/mol-175 g/mol (top note).

16. The composition as claimed in claim 1, wherein a mass ratio of component (A) to a total mass of component (B) is in a range of 1:2-25:1.

17. The composition as claimed in claim 5 wherein component (B) comprises
(B)(i) one or more fragrance substances with a molecular weight in the range of 120 g/mol-155 g/mol.

18. The composition as claimed in claim 6, wherein component (C) comprises (C)(ii) dipropylene glycol.

19. The composition as claimed in claim 7 wherein component (D) is selected from the group consisting of glycerin, 1,2-propylene glycol, 1,2-butylene glycol, 1,3-butylene glycol and alkane diols with 5-12 C-atoms.

20. The topical cosmetic product as claimed in claim 8 comprising
(A) neopentyl glycol diisononanoate in a total quantity of 0.25-30 wt % and
(B) one or more fragrance substances in a quantity of 0.15-5 wt %, wherein component (B) comprises
(B)(i) one or more fragrance substances with a molecular weight in the range of 100 g/mol-175 g/mol (top note),
and/or
(B)(ii) one or more fragrance substances with a molecular weight in the range of greater than or equal to 190 g/mol (bottom note),
and/or
(C) or more cosmetically acceptable solubilizers for component (B) selected from the group consisting of
(C)(ii) dipropylene glycol, diethyl phthalate, triethyl citrate, isopropyl myristate and triacetin, wherein a total quantity of component (C) (ii) is up to 80 wt. % in relation to a total weight of component (B),
and/or
(D) one or more diols or triols with 3-12 C-atoms in a total quantity of 0.2-20 wt. % in relation to the total weight of the product.
